(19) United States
(12) Patent Application Publication
(10) Pub. No.: US 2023/0223155 A1
(43) Pub. Date: Jul. 13, 2023

(54) METHOD FOR EVALUATING DISTRIBUTION OF INFECTED PERSONS IN AREA

(71) Applicants: TSINGHUA UNIVERSITY, Beijing (CN); BEIJING GLOBAL SAFETY TECHNOLOGY CO., LTD., Beijing (CN)

(72) Inventors: Tao CHEN, Beijing (CN); Zhanhui SUN, Beijing (CN); Guofeng SU, Beijing (CN); Jiakun DAI, Beijing (CN); Lida HUANG, Beijing (CN); Xiaoli YAN, Beijing (CN)

(21) Appl. No.: 18/188,028

(22) Filed: Mar. 22, 2023

Related U.S. Application Data
(63) Continuation of application No. PCT/CN2021/109783, filed on Jul. 30, 2021.

(30) Foreign Application Priority Data
Sep. 23, 2020 (CN) 202011009826.6

(51) Int. Cl. G16H 50/80

(52) U.S. Cl. CPC G16H 50/80

(57) ABSTRACT

A method for evaluating a distribution of infected persons includes: acquiring an area to be processed, and dividing the area into a plurality of cells; acquiring personnel attribute information of each cell in each time window of a historical time period; determining a personnel flow probability from each cell to each other cell according to identifications of persons in each cell in each time window; evaluating, for each cell to be processed, personnel infection information of the cell after a time window according to current personnel infection information of the cell, a personnel flow probability from the cell to each other cell, a personnel flow probability from each other cell to the cell, and a preset epidemic model; and determining a distribution of infected persons in the area after the time window according to the personnel infection information of each cell to be processed after the time window.

### CROSS-REFERENCE TO RELATED APPLICATIONS

This application is a continuation of International Application No. PCT/CN2021/109783, filed Jul. 30, 2021, which claims priority to Chinese Patent Application No. 202011009826.6, filed Sep. 23, 2020, the entire disclosures of which are incorporated herein by reference.

### FIELD

The present disclosure relates to the technical field of data processing, and more particularly to a method and apparatus for evaluating a distribution of infected persons in an area.

### BACKGROUND

At present, a method for evaluating a distribution of infected persons in an area mainly adopts a cellular automata-based epidemic propagation model with inhomogeneity and mobility. In this epidemic propagation model, a location of each individual is regarded as a cell; the individual inhomogeneity, the individual mobility and an evolution rule are determined by taking the cell as a unit, and evolution is performed to determine the distribution of infected persons in the area after a time window. However, this method has large calculation amount, large consumption of calculation resources, and slow calculation speed.

### SUMMARY

Embodiments of a first aspect of the present disclosure propose a method for evaluating a distribution of infected persons in an area. The method includes: acquiring an area to be processed, and acquiring a plurality of cells obtained by dividing the area; acquiring personnel attribute information of each cell in each time window of a historical time period, in which the personnel attribute information of each cell includes identifications of persons in the cell; determining a personnel flow probability from each cell to each other cell according to the identifications of persons in each cell in each time window; evaluating, for each cell to be processed, personnel infection information of the cell to be processed after a time window according to current personnel infection information of the cell, a personnel flow probability from the cell to each other cell, a personnel flow probability from each other cell to the cell, and a preset epidemic model; and determining a distribution of infected persons in the area after the time window according to the personnel infection information of each cell to be processed after the time window.

Embodiments of a second aspect of the present disclosure propose an electronic device, which includes: a processor; a memory; and computer programs stored in the memory and executable by the processor. The processor, when executing the computer programs, causes the method for evaluating a distribution of infected persons in an area as described above to be implemented.

Embodiments of a third aspect of the present disclosure propose a computer-readable storage medium having stored therein computer programs that, when executed by a processor, cause the method for evaluating a distribution of infected persons in an area as described above to be implemented.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and/or other aspects and advantages of embodiments of the present disclosure will become apparent and more readily appreciated from the following descriptions made with reference to the drawings, in which:

FIG. 1 is a schematic flowchart of a method for evaluating a distribution of infected persons in an area according to embodiments of the present disclosure;

FIG. 2 is a schematic diagram showing a plurality of cells;

FIG. 3 is a schematic diagram showing a distribution of persons in an area to be processed in a time window;

FIG. 4 is a schematic flowchart of a method for evaluating a distribution of infected persons in an area according to embodiments of the present disclosure;

FIG. 5 is a schematic flowchart of a method for evaluating a distribution of infected persons in an area according to embodiments of the present disclosure;

FIG. 6 is a schematic block diagram of an apparatus for evaluating a distribution of infected persons in an area according to embodiments of the present disclosure; and

FIG. 7 is a schematic block diagram of an electronic device according to embodiments of the present disclosure.

### DETAILED DESCRIPTION

Reference will be made in detail to embodiments of the present disclosure, examples of which are illustrated in the accompanying drawings. The same or similar elements and the elements having same or similar functions are denoted by like reference numerals throughout the descriptions. The embodiments described herein with reference to drawings are explanatory, illustrative, and used to generally explain the present disclosure, but shall not be construed to limit the present disclosure.

Embodiments of the present disclosure seek to solve at least one of the problems existing in the related art to at least some extent.

For this, a first object of the present disclosure is to provide a method for evaluating a distribution of infected persons in an area, which is used to solve problems of heavy calculation burden, large consumption of calculation resources, and slow calculation speed in the related art.

A second object of the present disclosure is to provide an apparatus for evaluating a distribution of infected persons in an area.

A third object of the present disclosure is to provide an electronic device.

A fourth object of the present disclosure is to provide a computer-readable storage medium.

For achieving the above objects, embodiments of a first aspect of the present disclosure propose a method for evaluating a distribution of infected persons in an area. The method includes: acquiring an area to be processed, and acquiring a plurality of cells obtained by dividing the area; acquiring personnel attribute information of each cell in each time window of a historical time period, in which the personnel attribute information of each cell includes identifications of persons in the cell; determining a personnel flow probability from each cell to each other cell according to the identifications of persons in each cell in each time window; evaluating, for each cell to be processed, personnel infection information of the cell to be processed after a time window according to current personnel infection information of the cell, a personnel flow probability from the cell to each other cell, a personnel flow probability from each other cell to the cell, and a preset epidemic model; and determining a distribution of infected persons in the area after the time window according to the personnel infection information of each cell to be processed after the time window.

In an embodiment, determining the personnel flow probability from each cell to each other cell according to the identifications of persons in each cell in each time window includes: determining, for each cell, the number of persons co-existing in the cell and each other cell in the historical time period according to the identifications of persons in the cell in each time window and the identifications of persons in each other cell in each time window; acquiring a total number of persons existing in the cell, the total number of persons existing in the cell being a total number of persons that have existed in the cell in each time window; and determining the personnel flow probability from the cell to each other cell according to the number of persons co-existing in the cell and each other cell and the total number of persons existing in the cell.

In an embodiment, the historical time period includes n time windows, and determining, for each cell, the number of persons co-existing in the cell and each other cell in the historical time period according to the identifications of persons in the cell in each time window and the identification of persons in each other cell in each time window includes: obtaining, for each cell, a first union result of the cell by performing a union process on identifications of persons in the cell in first n−1 time windows of the historical time period; obtaining, for each other cell, a second union result of the other cell by performing a union process on identifications of persons in the other cell in last n−1 time windows of the historical time period; and determining the number of persons co-existing in the cell and each other cell in the historical time period according to the number of persons in an intersection of the first union result of the cell and the second union result of each other cell.

In an embodiment, evaluating, for each cell to be processed, the personnel infection information of the cell after the time window according to the current personnel infection information of the cell to be processed, the personnel flow probability from the cell to each other cell, the personnel flow probability from each other cell to the cell, and the preset epidemic model includes: evaluating first personnel infection change information of the cell in the time window according to the current personnel infection information of the cell and the preset epidemic model; evaluating second personnel infection change information between the cell and all other cells after the time window according to the current personnel infection information of the cell, current personnel infection information of each other cell, the personnel flow probability from the cell to each other cell, and the personnel flow probability from each other cell to the cell; and determining the personnel infection information of the cell after the time window according to the first personnel infection change information and the second personnel infection change information.

In an embodiment, evaluating the first personnel infection change information of the cell in the time window according to the current personnel infection information of the cell and the preset epidemic model includes: acquiring the current personnel infection information and current personnel number of each other cell; determining the current personnel number of the cell according to the current personnel infection information of the cell; and evaluating the first personnel infection change information of the cell in the time window in a personnel flowing case according to the current personnel infection information and the current personnel number of the cell, the current personnel infection information and the current personnel number of each other cell, and the preset epidemic model.

In an embodiment, the current personnel infection information includes identifications and infection states of persons. The evaluating the second personnel infection change information between the cell and all other cells after the time window according to the current personnel infection information of the cell, the current personnel infection information of each other cell, the personnel flow probability from the cell to each other cell, and the personnel flow probability from each other cell to the cell includes: determining the number of persons in each infection state in the cell according to the current personnel infection information of the cell; determining the number of persons in each infection state in each other cell according to the current personnel infection information of each other cell; inputting the number of persons in each infection state in the cell, the number of persons in each infection state in each other cell, the personnel flow probability from the cell to each other cell, and the personnel flow probability from each other cell to the cell into a personnel flow probability calculation formula to reversely solve the personnel flow probability calculation formula to obtain personnel infection change information between the cell and each other cell after the time window; and determining a sum of the personnel infection change information between the cell and each other cell after the time window as the second personnel infection change information between the cell and all other cells after the time window.

In an embodiment, after determining the distribution of infected persons in the area after the time window according to the personnel infection information of each cell to be processed after the time window, the method further includes: acquiring protection and rescue resource information of locations in the area; and allocating protection and rescue resources in the area according to the protection and rescue resource information of the locations in the area and the distribution of infected persons in the area after the time window.

In the method for evaluating a distribution of infected persons in an area according to embodiments of the present disclosure, the area to be processed is acquired, and the area is divided into the plurality of cells; the personnel attribute information of each cell in each time window of the historical time period is acquired, and the personnel attribute information of each cell includes identifications of persons in the cell; the personnel flow probability from each cell to each other cell is determined according to the identifications of persons in each cell in each time window; for each cell to be processed, the personnel infection information of the cell to be processed after a time window is evaluated according to the current personnel infection information of the cell, the personnel flow probability from the cell to each other cell, the personnel flow probability from each other cell to the cell, and the preset epidemic model; and the distribution of infected persons in the area after the time window is determined according to the personnel infection information of each cell to be processed after the time window. In this way, by dividing the area into the plurality of cells, rather than using individuals as cells, the personnel infection information of each cell to be processed after a time window can be determined according to the personnel infection change information of each cell and the personnel infection change information among cells after the time window, with small calculation amount, less consumption of calculation resources and fast calculation speed.

For achieving the above objects, embodiments of a second aspect of the present disclosure propose an apparatus for evaluating a distribution of infected persons in an area. The apparatus includes: a first acquiring module, configured to acquire an area to be processed, and acquire a plurality of cells obtained by dividing the area; a second acquiring module, configured to acquire personnel attribute information of each cell in each time window of a historical time period, in which the personnel attribute information of each cell includes identifications of persons in the cell; a first determining module, configured to determine a personnel flow probability from each cell to each other cell according to the identifications of persons in each cell in each time window; an evaluating module, configured to evaluate, for each cell to be processed, personnel infection information of the cell to be processed after a time window according to current personnel infection information of the cell, a personnel flow probability from the cell to each other cell, a personnel flow probability from each other cell to the cell, and a preset epidemic model; and a second determining module, configured to determine a distribution of infected persons in the area after the time window according to the personnel infection information of each cell to be processed after the time window.

In an embodiment, the first determining module is further configured to: determine, for each cell, the number of persons co-existing in the cell and each other cell in the historical time period according to the identifications of persons in the cell in each time window and the identifications of persons in each other cell in each time window; acquire a total number of persons existing in the cell, wherein the total number of persons existing in the cell is a total number of persons that have existed in the cell in each time window; and determine the personnel flow probability from the cell to each other cell according to the number of persons co-existing in the cell and each other cell and the total number of persons existing in the cell.

In an embodiment, the historical time period includes n time windows, and the first determining module is further configured to: obtain, for each cell, a first union result of the cell by performing a union process on identifications of persons in the cell in first n−1 time windows of the historical time period; obtain, for each other cell, a second union result of the other cell by performing a union process on identifications of persons in the other cell in last n−1 time windows of the historical time period; and determine the number of persons co-existing in the cell and each other cell in the historical time period according to the number of persons in an intersection of the first union result of the cell and the second union result of each other cell.

In an embodiment, the evaluating module is further configured to: evaluate first personnel infection change information of the cell in the time window according to the current personnel infection information of the cell and the preset epidemic model; evaluate second personnel infection change information between the cell and all other cells after the time window according to the current personnel infection information of the cell, current personnel infection information of each other cell, the personnel flow probability from the cell to each other cell, and the personnel flow probability from each other cell to the cell; and determine the personnel infection information of the cell after the time window according to the first personnel infection change information and the second personnel infection change information.

In an embodiment, the evaluating module is further configured to: acquire the current personnel infection information and current personnel number of each other cell; determine the current personnel number of the cell according to the current personnel infection information of the cell; and evaluate the first personnel infection change information of the cell in the time window in a personnel flowing case according to the current personnel infection information and the current personnel number of the cell, the current personnel infection information and the current personnel number of each other cell, and the preset epidemic model.

In an embodiment, the current personnel infection information includes identifications and infection states of persons. The evaluating module is further configured to: determine the number of persons in each infection state in the cell according to the current personnel infection information of the cell; determine the number of persons in each infection state in each other cell according to the current personnel infection information of each other cell; input the number of persons in each infection state in the cell, the number of persons in each infection state in each other cell, the personnel flow probability from the cell to each other cell, and the personnel flow probability from each other cell to the cell into a personnel flow probability calculation formula to reversely solve the personnel flow probability calculation formula to obtain personnel infection change information between the cell and each other cell after the time window; and determine a sum of the personnel infection change information between the cell and each other cell after the time window as the second personnel infection change information between the cell and all other cells after the time window.

In an embodiment, the apparatus further includes: a third acquiring module, configured to acquire protection and rescue resource information of locations in the area; and an allocating module, configured to allocate protection and rescue resources in the area according to the protection and rescue resource information of the locations in the area and the distribution of infected persons in the area after the time window.

In the apparatus for evaluating a distribution of infected persons in an area according to embodiments of the present disclosure, the area to be processed is acquired and divided into the plurality of cells; the personnel attribute information of each cell in each time window of the historical time period is acquired, and the personnel attribute information of each cell includes identifications of persons in the cell; the personnel flow probability from each cell to each other cell is determined according to the identifications of persons in each cell in each time window; for each cell to be processed, the personnel infection information of the cell to be processed after a time window is evaluated according to the current personnel infection information of the cell, the personnel flow probability from the cell to each other cell, the personnel flow probability from each other cell to the cell, and the preset epidemic model; and the distribution of infected persons in the area after the time window is determined according to the personnel infection information of each cell to be processed after the time window. In this way, by dividing the area into the plurality of cells, rather than using individuals as cells, the personnel infection information of each cell to be processed after a time window can be determined according to the personnel infection change information of each cell and the personnel infection change information among cells after the time window, with small calculation amount, less consumption of calculation resources and fast calculation speed.

Embodiments of a third aspect of the present disclosure propose an electronic device, which includes: a processor; a memory; and computer programs stored in the memory and executable by the processor. The processor, when executing the computer programs, causes the method for evaluating a distribution of infected persons in an area as described above to be implemented.

Embodiments of a fourth aspect of the present disclosure propose a computer-readable storage medium having stored therein computer programs that, when executed by a processor, cause the method for evaluating a distribution of infected persons in an area as described above to be implemented.

In the following, methods and apparatuses for evaluating a distribution of infected persons in an area according to embodiments of the present disclosure will be described in detail referring to the accompanying drawings.

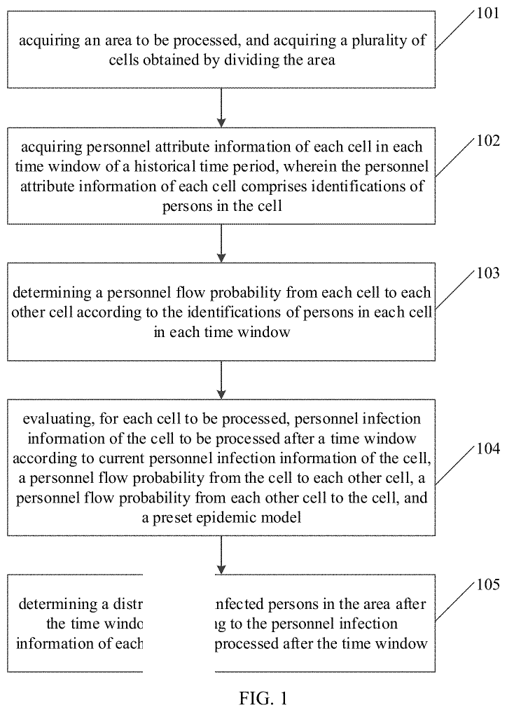

FIG. 1 is a schematic flowchart of a method for evaluating a distribution of infected persons in an area according to embodiments of the present disclosure. As shown in FIG. 1, the method includes the following steps.

At step 101, an area to be processed is acquired, and the area is divided into a plurality of cells.

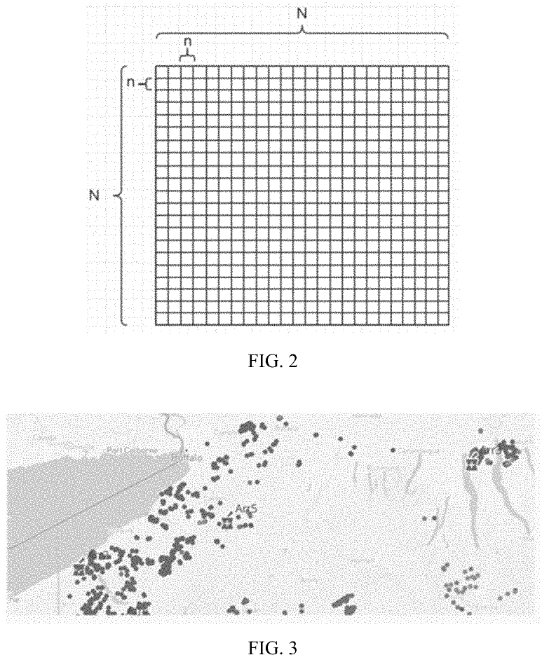

In embodiments of the present disclosure, the area to be processed may be any area, such as a county, a city, or a district of a city, such as Chaoyang District, Haidian District, and the like. The number of cells obtained by dividing the area may be determined according to the total number of persons in the area, the size of the area, the activity of the persons in the area and the calculation amount. FIG. 2 is a schematic diagram showing a plurality of cells. In FIG. 2, the area to be processed may be an area with a size of N×N (km), and may be divided into a plurality of cells, with each cell having a size of n×n (km), so the number of cells is (N/n)×(N/n).

At step 102, personnel attribute information of each cell in each time window of a historical time period is acquired. The personnel attribute information of each cell includes identifications of persons in the cell.

In embodiments of the present disclosure, the historical time period, for example, is a period from 8:00 to 18:00 in a day, and the historical time period may be divided into five time windows, which are a time window from 8:00 to 10:00, a time window from 10:00 to 12:00, a time window from 12:00 to 14:00, a time window from 14:00 to 16:00, and a time window from 16:00 to 18:00, for example.

In embodiments of the present disclosure, basically every person has a mobile phone at present, and a mobile phone operator can obtain location information of a person in real time. Therefore, location information of each mobile phone may be acquired by the mobile phone operator, then location information of users correspond to respective mobile phones may be acquired, so as to determine for each user a cell where the user is located. The identification of a person in a cell may be, for example, a mobile phone number of the corresponding user, the user name, a serial number of a device used by the user, or other parameter that can uniquely identify the user.

In embodiments of the present disclosure, within a time window, identifications of users whose respective location information is located in a cell may be collected in real time, and identifications of users collected in each time point are merged to obtain the personnel attribute information of the cell in the time window.

In embodiments of the present disclosure, the number of persons in a cell in a time window may be determined according to the personnel attribute information of the cell in the time window, and then the distribution of persons in the area to be process in the time window may be determined according to the number of persons in each cell in the time window. FIG. 3 shows a schematic diagram showing a distribution of persons in an area to be processed in a time window.

At step 103, a personnel flow probability from each cell to each other cell is determined according to the identifications of persons in each cell in each time window.

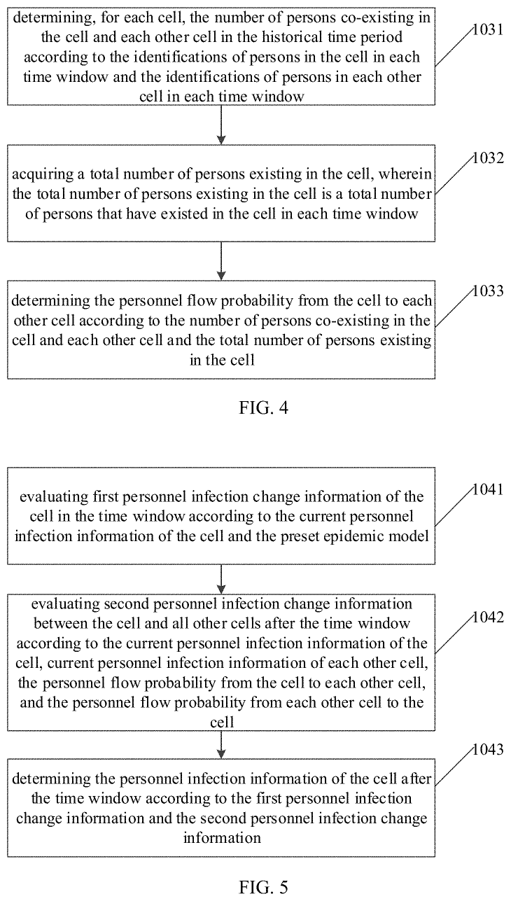

In embodiments of the present disclosure, the personnel flow probability indicates movements of persons among cells. The implementation of the step 103 by an apparatus for evaluating a distribution of infected persons in an area may refer to FIG. 4. As shown in FIG. 4, the step 103 may include the following steps.

At step 1031, for each cell, the number of persons co-existing in the cell and each other cell in the historical time period is determined according to the identifications of persons in the cell in each time window and the identifications of persons in each other cell in each time window.

In embodiments of the present disclosure, the historical time period may include n time windows. Since the movement of persons among cells takes time, so a person will not be in two cells at the same time, and it is difficult for a person to transfer from a cell to another cell in a very short time. Therefore, the implementation of the step 1031 by the apparatus for evaluating a distribution of infected persons in an area may include, for example, obtaining, for each cell, a first union result of the cell by performing a union process on identifications of persons in the cell in first n−1 time windows of the historical time period; obtaining, for each other cell, a second union result of the other cell by performing a union process on identifications of persons in the other cell in last n−1 time windows of the historical time period; and determining the number of persons co-existing in the cell and each other cell in the historical time period according to the number of persons in an intersection of the first union result of the cell and the second union result of each other cell.

In an embodiment of the present disclosure, n is 5, a cell is represented by (i, j), personnel attribute information of the cell (i, j) is represented by id (i, j), and personnel attribute information of the cell (i, j) in an n^{th} time window is represented by ldtn (i, j), personnel attribute information of an other cell in the n^{th} time window is represented by ldtn (p, k), then the number of persons co-existing in the cell (i, j) and the other cell (p, k) in the historical time period may be determined according to the following formula:

the number of persons co-existing in the cell(i,j) and the other cell(p,k) in the historical time period=Q((ldt1∪ldt2∪ldt3∪ldt4)(i,j)∩(ldt2∪ldt3∪ldt4∪ldt5)(p,k)).

At step 1032, a total number of persons existing in the cell is acquired. The total number of persons existing in the cell is a total number of persons that have existed in the cell in each time window.

In embodiments of the present disclosure, illustration is made with reference to the above example, and the total number of persons existing in the cell may be the total number of persons that have existed in the cell within 5 time windows. In addition, since the movement of persons among cells takes time, the personnel attribute information of the cell in the fifth time window does not make contribution to the calculation of the personnel flow probability. Therefore, in order to further improve the accuracy of the personnel flow probability, the personnel flow probability may be determined according to the personnel attribute information of the cell in the first n−1 time windows. In an example where n is 5, the total number of persons existing in the cell may be determined according to the following formula:

the total number of persons existing in the cell=Q((ldt1∪ldt2∪ldt3∪ldt4)(i,j)).

At step 1033, the personnel flow probability from the cell to each other cell is determined according to the number of persons co-existing in the cell and each other cell and the total number of persons existing in the cell.

In embodiments of the present disclosure, illustration is made with reference to the above example where n is 5, the personnel flow probability from the cell (i, j) to the other cell (p, k) may be determined according to the following formula:

R(i,j),(p,k)Q((ldt1∪ldt2∪ldt3∪ldt4)(i,j)∩(ldt2∪ldt3∪ldt4∪ldt5)(p,k))/Q((ldt1∪ldt2∪ldt3∪ldt4)(i,j)),

where R(i, j), (p, k) represents the personnel flow probability from the cell (i, j) to the other cell (p, k).

At step 104, for each cell to be processed, personnel infection information of the cell after a time window is evaluated according to current personnel infection information of the cell, a personnel flow probability from the cell to each other cell, a personnel flow probability from each other cell to the cell, and a preset epidemic model.

In embodiments of the present disclosure, the current personnel infection information may include identifications and infection states of persons. First personnel infection change information of the cell to be processed in the time window may be evaluated according to the identifications and infection states of persons in the cell to be processed and the preset epidemic model. Second personnel infection change information between the cell to be processed and other cells may be evaluated according to the current personnel infection information of the cell to be processed, the personnel flow probability from the cell to be processed to each other cell, and the personnel flow probability from each other cell to the cell to be processed. The personnel infection information of the cell to be processed after the time window may be determined according to the first personnel infection change information, the second personnel infection change information, and the current personnel infection information.

At step 105, a distribution of infected persons in the area after the time window is determined according to the personnel infection information of each cell to be processed after the time window.

In embodiments of the present disclosure, after the step 105, the apparatus for evaluating a distribution of infected persons in an area may further perform the following operation: acquiring protection and rescue resource information of locations in the area to be processed; and allocating protection and rescue resources in the area according to the protection and rescue resource information of the locations in the area and the distribution of infected persons in the area after the time window.

In the method for evaluating a distribution of infected persons in an area according to embodiments of the present disclosure, the area to be processed is acquired, and the area is divided into the plurality of cells; the personnel attribute information of each cell in each time window of the historical time period is acquired, and the personnel attribute information of each cell includes identifications of persons in the cell; the personnel flow probability from each cell to each other cell is determined according to the identifications of persons in each cell in each time window; for each cell to be processed, the personnel infection information of the cell to be processed after a time window is evaluated according to the current personnel infection information of the cell, the personnel flow probability from the cell to each other cell, the personnel flow probability from each other cell to the cell, and the preset epidemic model; and the distribution of infected persons in the area after the time window is determined according to the personnel infection information of each cell to be processed after the time window. In this way, by dividing the area into the plurality of cells, rather than using individuals as cells, the personnel infection information of each cell to be processed after a time window can be determined according to the personnel infection change information of each cell and the personnel infection change information among cells after the time window, with small calculation amount, small consumption of the computing resources and fast calculation speed.

FIG. 5 is a schematic flowchart of a method for evaluating a distribution of infected persons in an area according to embodiments of the present disclosure. As shown in FIG. 5, on the basis of the embodiments shown in FIG. 1, the step 104 may include the following steps.

At step 1041, first personnel infection change information of the cell in the time window is evaluated according to the current personnel infection information of the cell and the preset epidemic model.

The implementation of the step 1041 by the apparatus for evaluating a distribution of infected persons in an area may include, for example, acquiring the current personnel infection information and current personnel number of each other cell; determining the current personnel number of the cell according to the current personnel infection information of the cell; and evaluating the first personnel infection change information of the cell in the time window in a personnel flowing case according to the current personnel infection information and the current personnel number of the cell, the current personnel infection information and the current personnel number of each other cell, and the preset epidemic model.

In embodiments of the present disclosure, in a first implementation scenario, the current personnel number of the cell may be the number of personnel identifications in the current personnel infection information of the cell. In a second implementation scenario, the current personnel number of the cell may be determined according to the personnel number of the cell in a previous time window, the personnel attribute information of the cell and other cells in each time window. For example, the current personnel number of the cell may be determined according to the following formula:

$$d {d}_{t n , ( i , j )} = d {d}_{t n - 1 , ( i , j )} + {\sum }_{( p , k ) = 0}^{n} Q ( l {d}_{t n , ( i , j )} ⋃ {ld}_{t n - 1 , ( p , k )} ) + {\sum }_{( p , k ) = 0}^{n} Q ( l {d}_{t n , ( p , k )} ⋃ {ld}_{t n - 1 , ( i , j )} ) ,$$

where dd_{tn-1,(i, j)} represents the personnel number of a cell (i, j) to be processed in a previous time window, dd_{tn,(i, j)} represents the current personnel number of the cell (i, j), Σ_{(p,k)=0}ⁿQ(ld_{tn,(i,j)} ∪ld_{tn-1,(p,k)}) represents the number of persons who are in a cell (p, k) in the previous time window and is currently in the cell (i, j), i.e., the number of persons who transfer from other cell to the cell (i, j), and Σ_{(p,k)=0}ⁿQ(ld_{tn,(p,k)} ∪ld_{tn-1,(p,k)}) represents the number of persons who leave the cell (i, j).

In embodiments of the present disclosure, the epidemic model mainly studies the transmission speed, spatial range, transmission route, dynamic mechanism and other issues of an infectious disease, to guide the effective prevention and control of the infectious disease. In embodiments of the present disclosure, the current personnel infection information of the cell to be processed may include: identifications and infection states of persons in the cell. The infection states includes susceptible, latent, infected and recovered. Among others, the susceptible may be represented by S, the latent may be represented by E, the infected may be represented by I, and the recovered may be represented by R.

A transformation probability from the susceptible to the latent may be represented by a, which is generally related to a density of infected persons in the cell, i.e., a ratio of the number of infected persons in the cell to the number of persons in the cell. A transformation probability p from the latent to the infected and a transformation probability y from the infected to the recovered are related to transmission parameters of the infectious disease.

In embodiments of the present disclosure, in a first implementation scenario, the epidemic model may determine change information of persons in each infection state in the cell after the time window only based on dynamic differential equations, the above three transformation probabilities, the current number of susceptible persons, the current number of latent persons, the current number of infected persons and the current number of recovered persons in the cell input, without consideration of personnel flow among cells. The epidemic model determines the change information of persons in each infection state in the cell after the time window based on the following formulas:

$$\frac{d {S}_{( i , i )}}{d t} = - α \frac{{S}_{( i , j )}}{d {d}_{( i , j )}} {I}_{t n , ( i , j )} ;$$

$$\frac{d {E}_{( i , j )}}{d t} = α \frac{{S}_{( i , j )}}{d {d}_{( i , j )}} {I}_{( i , j )} - β {E}_{( i , j )} ;$$

$$\frac{d {I}_{( i , j )}}{d t} = β {E}_{( i , j )} - γ {I}_{( i , j )} ; and$$

$$\frac{d {R}_{( i , j )}}{d t} = γ {I}_{( i , j )} ,$$

where S_{(i, j)} represents the number of susceptible persons in the cell in the time window, I_{(i, j)} represents the number of infected persons in the cell in the time window, E_{(i, j)} represents the number of latent persons in the cell in the time window, R_{(i, j)} represents the number of recovered persons in the cell in the time window, the subscript tn of I_{(i, j)} represents an n^{th} time window, and dd_{(i, j)} represents the number of persons in the cell in the time window. When predicting the change information of persons in each infection state in the cell after 1 time window from the current time point, it may be predicted based on the current number of susceptible persons, the current number of latent persons, the current number of infected persons and the current number of recovered persons in the cell.

In embodiments of the present disclosure, in a second implementation scenario, under consideration of personnel flow among cells, the epidemic model may determine change information of persons in each infection state in the cell after the time window based on dynamic differential equations, the above three transformation probabilities, personnel flow situations among cells, the current number of susceptible persons, the current number of latent persons, the current number of infected persons and the current number of recovered persons in the cell input. The epidemic model determines the change information of persons in each infection state in the cell after the time window

$${\overline{S}}_{t n , ( i , j )} = {S}_{t n , ( i , j )} + {\sum }_{( p , k ) = 0}^{n} S ( p , k ) / {dd}_{tn - 1} ( p , k ) Q ( l {d}_{tn , ( i , j )} ⋃ {ld}_{tn - 1 , ( p , k )} ) + S ( i , j ) / {dd}_{tn - 1} ( i , j ) {\sum }_{( p , k ) = 0}^{n} Q ( l {d}_{tn , ( p , k )} ⋃ {ld}_{tn - 1 , ( i , j )} ) ;$$

$${\overline{E}}_{t n , ( i , j )} = {E}_{t n , ( i , j )} + {\sum }_{( p , k ) = 0}^{n} E ( p , k ) / {dd}_{tn - 1} ( p , k ) Q ( l {d}_{tn , ( i , j )} ⋃ {ld}_{tn - 1 , ( p , k )} ) + E ( i , j ) / {dd}_{tn - 1} ( i , j ) {\sum }_{( p , k ) = 0}^{n} Q ( l {d}_{tn , ( p , k )} ⋃ {ld}_{tn - 1 , ( i , j )} ) ;$$

$${\overline{I}}_{t n , ( i , j )} = {I}_{t n , ( i , j )} + {\sum }_{( p , k ) = 0}^{n} I ( p , k ) / {dd}_{tn - 1} ( p , k ) Q ( l {d}_{tn , ( i , j )} ⋃ {ld}_{tn - 1 , ( p , k )} ) + I ( i , j ) / {dd}_{tn - 1} ( i , j ) {\sum }_{( p , k ) = 0}^{n} Q ( l {d}_{tn , ( p , k )} ⋃ {ld}_{tn - 1 , ( i , j )} ) ; and$$

$${\overline{R}}_{t n , ( i , j )} = {R}_{t n , ( i , j )} + {\sum }_{( p , k ) = 0}^{n} R ( p , k ) / {dd}_{tn - 1} ( p , k ) Q ( l {d}_{tn , ( i , j )} ⋃ {ld}_{tn - 1 , ( p , k )} ) + R ( i , j ) / {dd}_{tn - 1} ( i , j ) {\sum }_{( p , k ) = 0}^{n} Q ( l {d}_{tn , ( p , k )} ⋃ {ld}_{tn - 1 , ( i , j )} ) ,$$

where a difference between S̅_{tn(i,j)} and S_{tn(i,j)} represents variation of susceptible persons in the first personnel infection change information of the cell (i, j) after 1 time window; a difference between Ē_{tn(i,j)} and E_{tn(i,j)} represents variation of latent persons in the first personnel infection change information of the cell (i,j) after 1 time window; a difference between Ī_{tn(i,j)} and I_{tn(i,j)} represents variation of infected persons in the first personnel infection change information of the cell (i, j) after 1 time window; and a difference between R̅_{tn(i,j)} and R_{tn(i,j)} represents variation of recovered persons in the first personnel infection change information of the cell (i, j) after 1 time window.

At S1042, second personnel infection change information between the cell and all other cells after the time window is evaluated according to the current personnel infection information of the cell, current personnel infection information of each other cell, the personnel flow probability from the cell to each other cell, and the personnel flow probability from each other cell to the cell.

In embodiments of the present disclosure, the current personnel infection information includes identifications and infection states of persons in the corresponding cell. Correspondingly, the implementation of the step 1042 by the apparatus for evaluating a distribution of infected persons in an area may include, for example, determining the number of persons in each infection state in the cell according to the current personnel infection information of the cell; determining the number of persons in each infection state in each other cell according to the current personnel infection information of each other cell; inputting the number of persons in each infection state in the cell, the number of persons in each infection state in each other cell, the personnel flow probability from the cell to each other cell, and the personnel flow probability from each other cell to the cell into a personnel flow probability calculation formula to reversely solve the personnel flow probability calculation formula to obtain personnel infection change information between the cell and each other cell after the time window; and determining a sum of the personnel infection change information between the cell and each other cell after the time window as the second personnel infection change information between the cell and all other cells after the time window.

In embodiments of the present disclosure, illustrations are made with reference to an example where a cell to be processed is represented by a cell (i, j), and other cells are represented by cells (p, k)₁, (p, k)₂, . . . , (p, k)ₙ, respectively. The determination of personnel infection change information between the cell (i, j) and the other cell (p, k)₁ may include: inputting the number of persons in each infection state in the cell (i, j), the number of persons in each infection state in the other cell (p, k)₁, the personnel flow probability from the cell (i, j) to the other cell (p, k)₁, and the personnel flow probability from the other cell (p, k)₁ to the cell (i, j) into the personnel flow probability calculation formula to reversely solve the personnel infection change information between the cell (i, j) and the other cell (p, k)₁ after the time window. Further, a sum of the personnel infection change information between the cell and each other cell after the time window is determined as the second personnel infection change information between the cell and all other cells after the time window.

At step 1043, the personnel infection information of the cell after the time window is determined according to the first personnel infection change information and the second personnel infection change information.

In embodiments of the present disclosure, the first personnel infection change information may include: the variation of susceptible persons, the variation of latent persons, the variation of infected persons, and the variation of recovered persons. The second personnel infection change information may include: the variation of susceptible persons, the variation of latent persons, the variation of infected persons, and the variation of recovered persons. For each infection state, a total variation of persons in the infection state may be obtained by adding the variation of persons in the infection state in the first personnel infection change information and the variation in the infection state in the second personnel infection change information, and the number of persons in the infection state after the change is determined according to the total variation of persons in the infection state and the number of persons in the infection state in the current personnel infection information. Further, based on the number of persons in each infection state after the change, the personnel infection information of the cell after the time window is determined.

In the method for evaluating a distribution of infected persons in an area according to embodiments of the present disclosure, the area to be processed is acquired, and the area is divided into the plurality of cells; the personnel attribute information of each cell in each time window of the historical time period is acquired, and the personnel attribute information of each cell includes identifications of persons in the cell; the personnel flow probability from each cell to each other cell is determined according to the identifications of persons in each cell in each time window; for each cell to be processed, the first personnel infection change information of the cell in the time window is evaluated according to the current personnel infection information of the cell and the preset epidemic model; the second personnel infection change information between the cell and all other cells after the time window is evaluated according to the current personnel infection information of the cell, the current personnel infection information of each other cell, the personnel flow probability from the cell to each other cell, and the personnel flow probability from each other cell to the cell; the personnel infection information of the cell after the time window is determined according to the first personnel infection change information and the second personnel infection change information; and the distribution of infected persons in the area after the time window is determined according to the personnel infection information of each cell to be processed after the time window. In this way, by dividing the area into the plurality of cells, rather than using individuals as cells, the personnel infection information of each cell to be processed after the time window can be determined according to the personnel infection change information of each cell and the personnel infection change information among cells after the time window, with small calculation amount, small consumption of the computing resources and fast calculation speed.

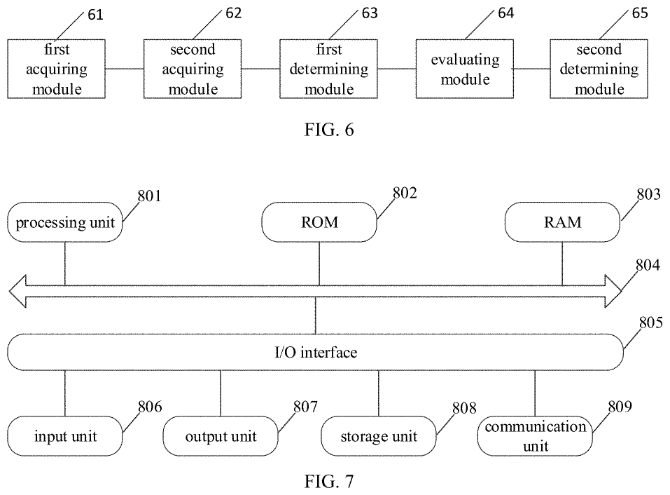

FIG. 6 is a schematic block diagram of an apparatus for evaluating a distribution of infected persons in an area according to embodiments of the present disclosure. As shown in FIG. 6, the apparatus includes a first acquiring module 61, a second acquiring module 62, a first determining module 63, an evaluating module 64, and a second determining module 65.

The first acquiring module 61 is configured to acquire an area to be processed, and acquire a plurality of cells obtained by dividing the area.

The second acquiring module 62 is configured to acquire personnel attribute information of each cell in each time window of a historical time period. The personnel attribute information of each cell includes identifications of persons in the cell.

The first determining module 63 is configured to determine a personnel flow probability from each cell to each other cell according to the identifications of persons in each cell in each time window.

The evaluating module 64 is configured to evaluate, for each cell to be processed, personnel infection information of the cell to be processed after a time window according to current personnel infection information of the cell, a personnel flow probability from the cell to each other cell, a personnel flow probability from each other cell to the cell, and a preset epidemic model.

The second determining module 65 is configured to determine a distribution of infected persons in the area after the time window according to the personnel infection information of each cell to be processed after the time window.

In an embodiment, the first determining module 63 is further configured to: determine, for each cell, the number of persons co-existing in the cell and each other cell in the historical time period according to the identifications of persons in the cell in each time window and the identifications of persons in each other cell in each time window; acquire a total number of persons existing in the cell, the total number of persons existing in the cell being a total number of persons that have existed in the cell in each time window; and determine the personnel flow probability from the cell to each other cell according to the number of persons co-existing in the cell and each other cell and the total number of persons existing in the cell.

In an embodiment, the historical time period includes n time windows, and the first determining module 63 is further configured to: obtain, for each cell, a first union result of the cell by performing a union process on identifications of persons in the cell in first n−1 time windows of the historical time period; obtain, for each other cell, a second union result of the other cell by performing a union process on identifications of persons in the other cell in last n−1 time windows of the historical time period; and determine the number of persons co-existing in the cell and each other cell in the historical time period according to the number of persons in an intersection of the first union result of the cell and the second union result of each other cell.

In an embodiment, the evaluating module 64 is further configured to: evaluate first personnel infection change information of the cell in the time window according to the current personnel infection information of the cell and the preset epidemic model; evaluate second personnel infection change information between the cell and all other cells after the time window according to the current personnel infection information of the cell, current personnel infection information of each other cell, the personnel flow probability from the cell to each other cell, and the personnel flow probability from each other cell to the cell; and determine the personnel infection information of the cell after the time window according to the first personnel infection change information and the second personnel infection change information.

In an embodiment, the evaluating module 64 is further configured to: acquire the current personnel infection information and current personnel number of each other cell; determine the current personnel number of the cell according to the current personnel infection information of the cell; and evaluate the first personnel infection change information of the cell in the time window in a personnel flowing case according to the current personnel infection information and the current personnel number of the cell, the current personnel infection information and the current personnel number of each other cell, and the preset epidemic model.

In an embodiment, the current personnel infection information includes identifications and infection states of persons. The evaluating module 64 is further configured to: determine the number of persons in each infection state in the cell according to the current personnel infection information of the cell; determine the number of persons in each infection state in each other cell according to the current personnel infection information of each other cell; input the number of persons in each infection state in the cell, the number of persons in each infection state in each other cell, the personnel flow probability from the cell to each other cell, and the personnel flow probability from each other cell to the cell into a personnel flow probability calculation formula to reversely solve the personnel flow probability calculation formula to obtain personnel infection change information between the cell and each other cell after the time window; and determine a sum of the personnel infection change information between the cell and each other cell after the time window as the second personnel infection change information between the cell and all other cells after the time window.

In an embodiment, the apparatus further includes a third acquiring module and an allocating module.

The third acquiring module is configured to acquire protection and rescue resource information of locations in the area.

The allocating module is configured to allocate protection and rescue resources in the area according to the protection and rescue resource information of the locations in the area and the distribution of infected persons in the area after the time window.

It should be noted that, for the descriptions of each module in the present disclosure, reference may be made to the method embodiments as shown in FIG. 1 to FIG. 5, which will not be described in detail here.

In the apparatus for evaluating a distribution of infected persons in an area according to embodiments of the present disclosure, the area to be processed is acquired and divided into the plurality of cells; the personnel attribute information of each cell in each time window of the historical time period is acquired, and the personnel attribute information of each cell includes identifications of persons in the cell; the personnel flow probability from each cell to each other cell is determined according to the identifications of persons in each cell in each time window; for each cell to be processed, the personnel infection information of the cell to be processed after a time window is evaluated according to the current personnel infection information of the cell, the personnel flow probability from the cell to each other cell, the personnel flow probability from each other cell to the cell, and the preset epidemic model; and the distribution of infected persons in the area after the time window is determined according to the personnel infection information of each cell to be processed after the time window. In this way, by dividing the area into the plurality of cells, rather than using individuals as cells, the personnel infection information of each cell to be processed after a time window can be determined according to the personnel infection change information of each cell and the personnel infection change information among cells after the time window, with small calculation amount, less consumption of calculation resources and fast calculation speed.

FIG. 7 is a schematic block diagram of an electronic device 800 suitable for implementing embodiments of the present disclosure. Referring to FIG. 7, the electronic device in embodiments of the present disclosure may include, but is not limited to, for example, a mobile terminal like a mobile phone, a laptop computer, a digital broadcast receiver, a personal digital assistant (PDA), a tablet computer (PAD), a portable multimedia player (PMP), a vehicle-mounted terminal (e.g., an in-vehicle navigation terminal) and the like, and a stationary terminal such as a digital TV, a desktop computer and the like. The electronic device shown in FIG. 7 is only an example, and should not impose any limitation on the functions and application scopes of embodiments of the present disclosure.

As shown in FIG. 7, the electronic device 800 may include a processing unit 801 (such as a central processing unit, a graphics processing unit), which can perform various suitable actions and processing according to a program stored in a read-only memory (ROM) 802 or a program loaded from a storage unit 808 to a random access memory (RAM) 803. In the RAM 803, various programs and data required for operations of the electronic device 800 may also be stored. The processing unit 801, the ROM 802 and the RAM 803 are connected to each other through a bus 804. An input/output (I/O) interface 805 is also connected to the bus 804.

In general, the following components may be connected to the I/O interface 805, including: an input unit 806, such as a touch screen, a touchpad, a keyboard, a mouse, a camera, a microphone, an accelerometer, a gyroscope, etc.; an output unit 807, such as a liquid crystal display (LCD), a speaker, a vibrator, etc.; a storage unit 808, such as a magnetic tape and a hard disk, etc.; and a communication unit 809. The communication unit 809 allows the electronic device 800 to exchange data with other devices through wireless or wired communication. Although FIG. 7 shows an electronic device 800 having various units, it should be understood that not all of the units as shown are required to be implemented or to be provided. Alternatively, electronic devices with more or less units may be implemented or provided.

In particular, according to embodiments of the present disclosure, the processes described above with reference to the flowcharts may be implemented as computer software programs. For example, embodiments of the present disclosure provide a computer program product. The computer program product includes a computer program carried on a computer-readable medium, and the computer program contains program codes for performing the method illustrated in the flowchart. In such embodiments, the computer program may be downloaded from the network via the communication unit 809 and installed, or installed from the storage unit 808 or the ROM 802. When the computer program is executed by the processing unit 801, the above-mentioned functions defined in the methods according to embodiments of the present disclosure are executed.

It should be noted that, the machine-readable medium as described above may be a machine-readable signal medium, a machine-readable storage medium, or any combinations thereof. The machine-readable storage medium may include, but is not limited to, an electronic, magnetic, optical, electromagnetic, infrared or semiconductor system, apparatus or device or any suitable combination of the foregoing. More specific examples of the machine-readable storage medium may include, but is not limited to, electrical connection having one or more wires, a portable computer disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device or any suitable combination of the foregoing. In the present disclosure, the computer-readable storage medium may be any tangible medium that contains or stores a program that can be used by or in combination with an instruction execution system, apparatus, or device. In the present disclosure, however, the computer-readable signal medium may include a data signal propagated in baseband or as a part of a carrier wave with a computer-readable program code carried thereon. The propagated data signal may be in various forms, including but not limited to an electromagnetic signal, an optical signal, or any suitable combination of the foregoing. The computer-readable signal medium may also be any computer-readable medium other than the computer-readable storage medium, and the computer-readable signal medium can transmit, propagate, or transport programs for use by or in connection with the instruction execution system, apparatus, or device. The program code carried on the computer readable medium may be transmitted using any suitable medium including, but not limited to, an electrical wire, an optical cable, a radio frequency (RF), etc., or any suitable combination of the foregoing.

The above-mentioned computer-readable medium may be included in the above-mentioned electronic device; or may exist alone without being assembled into the electronic device.

The above-mentioned computer-readable medium carries one or more programs that, when executed by the electronic device, cause the electronic device to: acquire an area to be processed, and acquire a plurality of cells obtained by dividing the area; acquire personnel attribute information of each cell in each time window of a historical time period, in which the personnel attribute information of each cell includes identifications of persons in the cell; determine a personnel flow probability from each cell to each other cell according to the identifications of persons in each cell in each time window; evaluate, for each cell to be processed, personnel infection information of the cell to be processed after a time window according to current personnel infection information of the cell, a personnel flow probability from the cell to each other cell, a personnel flow probability from each other cell to the cell, and a preset epidemic model; and determine a distribution of infected persons in the area after the time window according to the personnel infection information of each cell to be processed after the time window.

The computer program code for carrying out operations of embodiments of the present disclosure may be written in one or more programming languages, or a combination thereof. The programming language includes an object oriented programming language, such as Java, Smalltalk, C++, as well as conventional procedural programming language, such as “C” language or similar programming language. The program code may be executed entirely on a user's computer, partly on the user's computer, as a separate software package, partly on the user's computer and partly on a remote computer, or entirely on the remote computer or server. In a case of the remote computer, the remote computer may be connected to the user's computer or an external computer (such as using an Internet service provider to connect over the Internet) through any kind of network, including a local area network (LAN) or a wide area network (WAN).

Embodiments of the present disclosure further provide a computer-readable storage medium having stored therein computer programs that, when executed by a processor, cause the method for evaluating a distribution of infected persons in an area as described above to be implemented.

Embodiments of the present disclosure further provide a computer program product including instructs that, when executed by a processor, cause the method for evaluating a distribution of infected persons in an area as described above to be implemented.

Reference throughout this specification to “an embodiment,” “some embodiments,” “an example,” “a specific example,” or “some examples,” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example,” “in an example,” “in a specific example,” or “in some examples,” in various places throughout this specification are not necessarily referring to the same embodiment or example of the present disclosure. Furthermore, the particular features, structures, materials, or characteristics may be combined in any suitable manner in one or more embodiments or examples. In addition, in the absence of contradiction, different embodiments or examples described in this specification, or features of different embodiments or examples may be combined by those skilled in the art.

In addition, terms such as “first” and “second” are used herein for purposes of description and are not intended to indicate or imply relative importance or significance or to imply the number of indicated technical features. Thus, the feature defined with “first” and “second” may explicitly or implicitly comprise one or more of this feature. In the description of the present invention, the phrase of “a plurality of” means two or more than two, such as two or three, unless specified otherwise.

Any process or method described in a flowchart or described herein in other ways may be understood to include one or more modules, segments or portions of codes of executable instructions for achieving specific logical functions or steps in the process, and the scope of a preferred embodiment of the present disclosure includes other implementations, in which the order of execution is different from what is shown or discussed, including executing functions in a substantially simultaneous manner or in an opposite order according to the related functions. These and other aspects should be understood by those skilled in the art to which embodiments of the present disclosure belong.

The logic and/or step shown in the flowchart or described in other manners herein, for example, a particular sequence table of executable instructions for realizing the logical function, may be specifically achieved in any computer readable medium to be used by the instruction execution system, device or equipment (such as the system based on computers, the system comprising processors or other systems capable of obtaining the instruction from the instruction execution system, device and equipment and executing the instruction), or to be used in combination with the instruction execution system, device and equipment. As to the specification, “the computer readable medium” may be any device adaptive for including, storing, communicating, propagating or transferring programs to be used by or in combination with the instruction execution system, device or equipment. More specific examples of the computer readable medium include but are not limited to: an electronic connection (an electronic device) with one or more wires, a portable computer enclosure (a magnetic device), a random access memory (RAM), a read only memory (ROM), an erasable programmable read-only memory (EPROM or a flash memory), an optical fiber device and a portable compact disk read-only memory (CDROM). In addition, the computer readable medium may even be a paper or other appropriate medium capable of printing programs thereon, this is because, for example, the paper or other appropriate medium may be optically scanned and then edited, decrypted or processed with other appropriate methods when necessary to obtain the programs in an electric manner, and then the programs may be stored in the computer memories.

It should be understood that each part of the present disclosure may be realized by the hardware, software, firmware or their combination. In the above embodiments, a plurality of steps or methods may be realized by the software or firmware stored in the memory and executed by the appropriate instruction execution system. For example, if it is realized by the hardware, likewise in another embodiment, the steps or methods may be realized by one or a combination of the following techniques known in the art: a discrete logic circuit having a logic gate circuit for realizing a logic function of a data signal, an application-specific integrated circuit having an appropriate combination logic gate circuit, a programmable gate array (PGA), a field programmable gate array (FPGA), etc.

It can be understood by those ordinarily skilled in the art that all or part of the steps in the method of the above embodiments can be implemented by instructing related hardware via programs, the program may be stored in a computer readable storage medium, and the program includes one step or combinations of the steps of the method when the program is executed.

In addition, each functional unit in embodiments of the present disclosure may be integrated in one progressing module, or each functional unit exists as an independent unit, or two or more functional units may be integrated in one module. The integrated module can be embodied in hardware, or software function module. If the integrated module is embodied in software function module and sold or used as an independent product, it can be stored in the computer readable storage medium.

The readable storage medium described above may be read-only memories, magnetic disks, or optical disks. Although some embodiments of the present disclosure have been shown and described above, it would be appreciated by those skilled in the art that the above embodiments are illustrative and explanatory, and cannot be construed to limit the present disclosure. Changes, alternatives, modifications and variants can be made in embodiments by those skilled in the art without departing from the scope of the present disclosure.

What is claimed is:

1. A method for evaluating a distribution of infected persons in an area, comprising:
acquiring an area to be processed, and acquiring a plurality of cells obtained by dividing the area;
acquiring personnel attribute information of each cell in each time window of a historical time period, wherein the personnel attribute information of each cell comprises identifications of persons in the cell;
determining a personnel flow probability from each cell to each other cell according to the identifications of persons in each cell in each time window;
evaluating, for each cell to be processed, personnel infection information of the cell to be processed after a time window according to current personnel infection information of the cell, a personnel flow probability from the cell to each other cell, a personnel flow probability from each other cell to the cell, and a preset epidemic model; and
determining a distribution of infected persons in the area after the time window according to the personnel infection information of each cell to be processed after the time window;
wherein the personnel flow probability indicates a personnel flow situation among cells, and determining the personnel flow probability from each cell to each other cell according to the identifications of persons in each cell in each time window comprises:
determining, for each cell, the number of persons co-existing in the cell and each other cell in the historical time period according to the identifications of persons in the cell in each time window and the identifications of persons in each other cell in each time window;
acquiring a total number of persons existing in the cell, wherein the total number of persons existing in the cell is a total number of persons that have existed in the cell in each time window; and
determining the personnel flow probability from the cell to each other cell according to the number of persons co-existing in the cell and each other cell and the total number of persons existing in the cell,
wherein evaluating, for each cell to be processed, the personnel infection information of the cell after the time window according to the current personnel infection information of the cell to be processed, the personnel flow probability from the cell to each other cell, the personnel flow probability from each other cell to the cell, and the preset epidemic model comprises:
evaluating first personnel infection change information of the cell in the time window according to the current personnel infection information of the cell and the preset epidemic model;
evaluating second personnel infection change information between the cell and all other cells after the time window according to the current personnel infection information of the cell, current personnel infection information of each other cell, the personnel flow probability from the cell to each other cell, and the personnel flow probability from each other cell to the cell; and
determining the personnel infection information of the cell after the time window according to the first personnel infection change information and the second personnel infection change information;
wherein evaluating the first personnel infection change information of the cell in the time window according to the current personnel infection information of the cell and the preset epidemic model comprises:
acquiring the current personnel infection information and current personnel number of each other cell;
determining the current personnel number of the cell according to the current personnel infection information of the cell; and
evaluating the first personnel infection change information of the cell in the time window in a personnel flowing case according to the current personnel infection information and the current personnel number of the cell, the current personnel infection information and the current personnel number of each other cell, and the preset epidemic model;
wherein the current personnel infection information comprises identifications and infection states of persons;
wherein evaluating the second personnel infection change information between the cell and all other cells after the time window according to the current personnel infection information of the cell, the current personnel infection information of each other cell, the personnel flow probability from the cell to each other cell, and the personnel flow probability from each other cell to the cell comprises:
determining the number of persons in each infection state in the cell according to the current personnel infection information of the cell;
determining the number of persons in each infection state in each other cell according to the current personnel infection information of each other cell;
inputting the number of persons in each infection state in the cell, the number of persons in each infection state in each other cell, the personnel flow probability from the cell to each other cell, and the personnel flow probability from each other cell to the cell into a personnel flow probability calculation formula to reversely solve the personnel flow probability calculation formula to obtain personnel infection change information between the cell and each other cell after the time window; and
determining a sum of the personnel infection change information between the cell and each other cell after the time window as the second personnel infection change information between the cell and all other cells after the time window.

2. The method according to claim 1, wherein the historical time period comprises n time windows,
wherein determining, for each cell, the number of persons co-existing in the cell and each other cell in the historical time period according to the identifications of persons in the cell in each time window and the identification of persons in each other cell in each time window comprises:
obtaining, for each cell, a first union result of the cell by performing a union process on identifications of persons in the cell in first n−1 time windows of the historical time period;
obtaining, for each other cell, a second union result of the other cell by performing a union process on identifications of persons in the other cell in last n−1 time windows of the historical time period; and
determining the number of persons co-existing in the cell and each other cell in the historical time period according to the number of persons in an intersection of the first union result of the cell and the second union result of each other cell.

3. The method according to claim 1, after determining the distribution of infected persons in the area after the time window according to the personnel infection information of each cell to be processed after the time window, further comprising:
acquiring protection and rescue resource information of locations in the area; and
allocating protection and rescue resources in the area according to the protection and rescue resource information of the locations in the area and the distribution of infected persons in the area after the time window.

4. An electronic device, comprising:
a processor;
a memory; and
computer programs stored in the memory and executable by the processor;
wherein the processor, when executing the computer programs, causes a method for evaluating a distribution of infected persons in an area to be implemented, the method comprises:
acquiring an area to be processed, and acquiring a plurality of cells obtained by dividing the area;
acquiring personnel attribute information of each cell in each time window of a historical time period, wherein the personnel attribute information of each cell comprises identifications of persons in the cell;
determining a personnel flow probability from each cell to each other cell according to the identifications of persons in each cell in each time window;
evaluating, for each cell to be processed, personnel infection information of the cell to be processed after a time window according to current personnel infection information of the cell, a personnel flow probability from the cell to each other cell, a personnel flow probability from each other cell to the cell, and a preset epidemic model; and
determining a distribution of infected persons in the area after the time window according to the personnel infection information of each cell to be processed after the time window;
wherein the personnel flow probability indicates a personnel flow situation among cells, and determining the personnel flow probability from each cell to each other cell according to the identifications of persons in each cell in each time window comprises:
determining, for each cell, the number of persons co-existing in the cell and each other cell in the historical time period according to the identifications of persons in the cell in each time window and the identifications of persons in each other cell in each time window;
acquiring a total number of persons existing in the cell, wherein the total number of persons existing in the cell is a total number of persons that have existed in the cell in each time window; and
determining the personnel flow probability from the cell to each other cell according to the number of persons co-existing in the cell and each other cell and the total number of persons existing in the cell,
wherein evaluating, for each cell to be processed, the personnel infection information of the cell after the time window according to the current personnel infection information of the cell to be processed, the personnel flow probability from the cell to each other cell, the personnel flow probability from each other cell to the cell, and the preset epidemic model comprises:
evaluating first personnel infection change information of the cell in the time window according to the current personnel infection information of the cell and the preset epidemic model;
evaluating second personnel infection change information between the cell and all other cells after the time window according to the current personnel infection information of the cell, current personnel infection information of each other cell, the personnel flow probability from the cell to each other cell, and the personnel flow probability from each other cell to the cell; and
determining the personnel infection information of the cell after the time window according to the first personnel infection change information and the second personnel infection change information;
wherein evaluating the first personnel infection change information of the cell in the time window according to the current personnel infection information of the cell and the preset epidemic model comprises:
acquiring the current personnel infection information and current personnel number of each other cell;
determining the current personnel number of the cell according to the current personnel infection information of the cell; and
evaluating the first personnel infection change information of the cell in the time window in a personnel flowing case according to the current personnel infection information and the current personnel number of the cell, the current personnel infection information and the current personnel number of each other cell, and the preset epidemic model;
wherein the current personnel infection information comprises identifications and infection states of persons;
wherein evaluating the second personnel infection change information between the cell and all other cells after the time window according to the current personnel infection information of the cell, the current personnel infection information of each other cell, the personnel flow probability from the cell to each other cell, and the personnel flow probability from each other cell to the cell comprises:
determining the number of persons in each infection state in the cell according to the current personnel infection information of the cell;
determining the number of persons in each infection state in each other cell according to the current personnel infection information of each other cell;
inputting the number of persons in each infection state in the cell, the number of persons in each infection state in each other cell, the personnel flow probability from the cell to each other cell, and the personnel flow probability from each other cell to the cell into a personnel flow probability calculation formula to reversely solve the personnel flow probability calculation formula to obtain personnel infection change information between the cell and each other cell after the time window; and
determining a sum of the personnel infection change information between the cell and each other cell after the time window as the second personnel infection change information between the cell and all other cells after the time window.

5. The electronic device according to claim 4, wherein the historical time period comprises n time windows,
wherein determining, for each cell, the number of persons co-existing in the cell and each other cell in the historical time period according to the identifications of persons in the cell in each time window and the identification of persons in each other cell in each time window comprises:
obtaining, for each cell, a first union result of the cell by performing a union process on identifications of persons in the cell in first n−1 time windows of the historical time period;
obtaining, for each other cell, a second union result of the other cell by performing a union process on identifications of persons in the other cell in last n−1 time windows of the historical time period; and
determining the number of persons co-existing in the cell and each other cell in the historical time period according to the number of persons in an intersection of the first union result of the cell and the second union result of each other cell.

6. The electronic device according to claim 4, after determining the distribution of infected persons in the area after the time window according to the personnel infection information of each cell to be processed after the time window, further comprising:
acquiring protection and rescue resource information of locations in the area; and
allocating protection and rescue resources in the area according to the protection and rescue resource information of the locations in the area and the distribution of infected persons in the area after the time window.

7. A non-transitory computer-readable storage medium having stored therein computer programs that, when executed by a processor, cause a method for evaluating a distribution of infected persons in an area to be implemented, the method comprises:
acquiring an area to be processed, and acquiring a plurality of cells obtained by dividing the area;
acquiring personnel attribute information of each cell in each time window of a historical time period, wherein the personnel attribute information of each cell comprises identifications of persons in the cell;
determining a personnel flow probability from each cell to each other cell according to the identifications of persons in each cell in each time window;
evaluating, for each cell to be processed, personnel infection information of the cell to be processed after a time window according to current personnel infection information of the cell, a personnel flow probability from the cell to each other cell, a personnel flow probability from each other cell to the cell, and a preset epidemic model; and
determining a distribution of infected persons in the area after the time window according to the personnel infection information of each cell to be processed after the time window;
wherein the personnel flow probability indicates a personnel flow situation among cells, and determining the personnel flow probability from each cell to each other cell according to the identifications of persons in each cell in each time window comprises:
determining, for each cell, the number of persons co-existing in the cell and each other cell in the historical time period according to the identifications of persons in the cell in each time window and the identifications of persons in each other cell in each time window;
acquiring a total number of persons existing in the cell, wherein the total number of persons existing in the cell is a total number of persons that have existed in the cell in each time window; and
determining the personnel flow probability from the cell to each other cell according to the number of persons co-existing in the cell and each other cell and the total number of persons existing in the cell,
wherein evaluating, for each cell to be processed, the personnel infection information of the cell after the time window according to the current personnel infection information of the cell to be processed, the personnel flow probability from the cell to each other cell, the personnel flow probability from each other cell to the cell, and the preset epidemic model comprises:
evaluating first personnel infection change information of the cell in the time window according to the current personnel infection information of the cell and the preset epidemic model;
evaluating second personnel infection change information between the cell and all other cells after the time window according to the current personnel infection information of the cell, current personnel infection information of each other cell, the personnel flow probability from the cell to each other cell, and the personnel flow probability from each other cell to the cell; and
determining the personnel infection information of the cell after the time window according to the first personnel infection change information and the second personnel infection change information;
wherein evaluating the first personnel infection change information of the cell in the time window according to the current personnel infection information of the cell and the preset epidemic model comprises:
acquiring the current personnel infection information and current personnel number of each other cell;
determining the current personnel number of the cell according to the current personnel infection information of the cell; and
evaluating the first personnel infection change information of the cell in the time window in a personnel flowing case according to the current personnel infection information and the current personnel number of the cell, the current personnel infection information and the current personnel number of each other cell, and the preset epidemic model;
wherein the current personnel infection information comprises identifications and infection states of persons;
wherein evaluating the second personnel infection change information between the cell and all other cells after the time window according to the current personnel infection information of the cell, the current personnel infection information of each other cell, the personnel flow probability from the cell to each other cell, and the personnel flow probability from each other cell to the cell comprises:
determining the number of persons in each infection state in the cell according to the current personnel infection information of the cell;
determining the number of persons in each infection state in each other cell according to the current personnel infection information of each other cell;
inputting the number of persons in each infection state in the cell, the number of persons in each infection state in each other cell, the personnel flow probability from the cell to each other cell, and the personnel flow probability from each other cell to the cell into a personnel flow probability calculation formula to reversely solve the personnel flow probability calculation formula to obtain personnel infection change information between the cell and each other cell after the time window; and
determining a sum of the personnel infection change information between the cell and each other cell after the time window as the second personnel infection change information between the cell and all other cells after the time window.

8. The non-transitory computer-readable storage medium according to claim 7, wherein the historical time period comprises n time windows,
wherein determining, for each cell, the number of persons co-existing in the cell and each other cell in the historical time period according to the identifications of persons in the cell in each time window and the identification of persons in each other cell in each time window comprises:
obtaining, for each cell, a first union result of the cell by performing a union process on identifications of persons in the cell in first n−1 time windows of the historical time period;
obtaining, for each other cell, a second union result of the other cell by performing a union process on identifications of persons in the other cell in last n−1 time windows of the historical time period; and
determining the number of persons co-existing in the cell and each other cell in the historical time period according to the number of persons in an intersection of the first union result of the cell and the second union result of each other cell.

9. The non-transitory computer-readable storage medium according to claim 7, after determining the distribution of infected persons in the area after the time window according to the personnel infection information of each cell to be processed after the time window, further comprising:
acquiring protection and rescue resource information of locations in the area; and
allocating protection and rescue resources in the area according to the protection and rescue resource information of the locations in the area and the distribution of infected persons in the area after the time window.
